(19) United States
(12) Plant Patent Application Publication
(10) Pub. No.: US 2002/0133857 P1
(43) Pub. Date: Sep. 19, 2002

(54) Alstroemeria plant named 'Isabel'

(75) Inventor: Robert Adrian Goemans, Chichester (GB)

Correspondence Address: FOLEY & LARDNER, 3000 K Street, N.W., Suite 500, Washington, DC, 20007-5109, US

(73) Assignee: Wulfinghoff Alstroemeria B.V.

(21) Appl. No.: 09/804,236

(22) Filed: Mar. 13, 2001

(51) Int. Cl. A01H 5/00

(52) U.S. Cl. PLT/309

(57) ABSTRACT

A new and distinct Alstroemeria plant named ‘Isabel’ characterized by growth height of 10-30 cm; suitable for pots with a diameter larger than 7 cm; bushy, dwarf growth habit; red flowers, yellow base with black stripes; and 4-6 week inflorescence longevity.

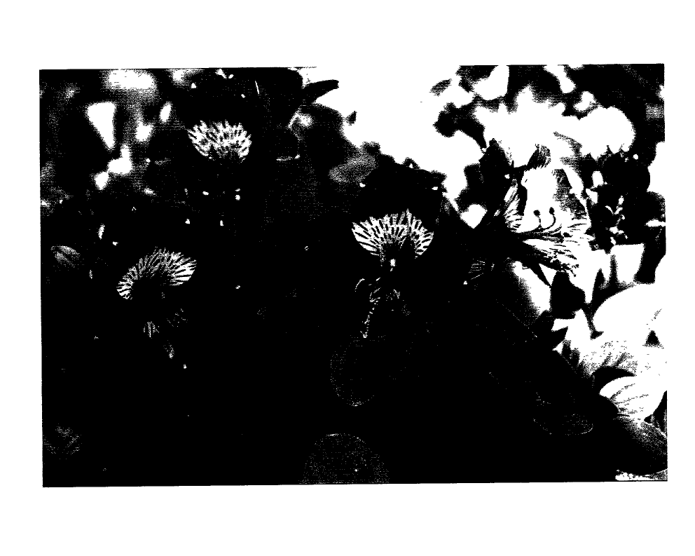

### BACKGROUND OF THE INVENTION

The present invention comprises a new and distinct cultivar of Alstroemeria plant, hereinafter referred to by the cultivar name ‘Isabel’.

The new cultivar,‘Isabel’, was originated by Robert Adrian Goemans from a hybridization made in a controlled breeding program in Chichester in Sussex, United Kingdom. The female parent was an unknown cultivar of unknown origin. The male parent of ‘Isabel’ was a selection of Butterfly.

‘Isabel’ was discovered and selected as one flowering plant within the progeny of the stated parentage by Robert Adrian Goemans in 1997 in a controlled environment in Chichester, United Kingdom. The first act of asexual reproduction of ‘Isabel’ was accomplished when vegetative cuttings were taken from the initial selection in May, 1998, in a controlled environment in Chichester, United Kingdom. Horticultural examination of selected units initiated in 1998, has demonstrated that the combination of characteristics as herein disclosed for ‘Isabel’ are firmly fixed and retained through successive generations of asexual reproduction.

### BRIEF DESCRIPTION OF THE INVENTION

The following traits have been repeatedly observed and are determined to be basic characteristics of which in combination distinguish this Alstroemeria as a new and distinct cultivar:

1. suitable for pots with a diameter larger than 7 cm;

2. growth height of 10-30 cm;

3. bushy, dwarf growth habit;

4. red flowers, yellow base with black stripes; and

5. 4-6 week inflorescence longevity.

‘Isabel’ has not been observed under all possible environmental conditions. The phenotype may vary significantly with variations in environment such as temperature, light intensity and day length, without any change in genotype. The following observations, measurements and comparisons describe plants grown in Chichester, Sussex, United Kingdom under greenhouse conditions which approximate those generally used in commercial practice.

There are no commercial cultivars known to the present inventor, similar in comparison to ‘Isabel’

### BRIEF DESCRIPTION OF THE DRAWINGS

The accompanying photograph shows a typical specimen of the new variety with colors as accurate as possible. Sheet 1 shows the flowers and foliage of ‘Isabel’.

### DETAILED BOTANICAL DESCRIPTION

In the following description, color references are made to The Royal Horticultural Society Colour Chart (RHS). The color values were determined in the afternoon on Sep. 16, 2000 under normal daylight at Wateringen, The Netherlands.

Classification:

Botanical.—Alstroemeria.

Commercial.—Alstroemeria CV ‘Isabel’.

Plant:

Form.—Pot Plant.

Height.—10-30 cm under Dutch greenhouse conditions.

Growth.—Bushy dwarf growth habit.

Foliage.—Quantity: 10-20 leaves on each stem, depending on growth stage Size of leaf: Length: 35-50 mm Width: 28-35 mm Shape of leaf: Oblong-ovate Color: Upper side: Dark green RHS 141 A to 141 B Underside: Light-green RHS 138 A.

Rhizomes.—Color: White RHS 158 A to 158 D Size: Length 10-50 mm; diameter 6-15 mm, depending on age.

Inflorescence:

Bud.—Form: Tubular pointed Diameter: 10-20 mm Length: 15-25 mm Peduncle: 20-25 mm medium-green RHS 144 D to 144 B Color: Red RHS 46 A to RHS 46 B.

Flower.—Size: Medium, length 40-60 mm; width 40-60 mm Borne: Medium number of branches in umbel. Shape: Broad obovate. Color: Upper surface: Red RHS 46B to RHS 46C Under surface: Yellow at base RHS 12 A to 13 A Other notations: Black stripes on all petals RHS 202 A.

Peduncle.—Length: 40-50 mm Color: Medium-green RHS 144 D to 144 B.

Lasting quality of the bloom.—4-6 weeks.

Reproductive Organs:

Stamens.—6.

Anthers.—Yellow RHS 13 A.

Filaments.—Red RHS 42 B to 44 B.

Pistils.—1, Red-pink. RHS 50 B to 50 C.

Stigma.—Pink-white RHS 36 A to 36 C.

I claim:

1. A new and distinct Alstroemeria plant named ‘Isabel’, substantially as illustrated and described herein.
